(19) United States
(12) Patent Application Publication
(10) Pub. No.: US 2025/0241620 A1
(43) Pub. Date: Jul. 31, 2025

(54) PUNCTURE SUPPORT APPARATUS, OPERATION METHOD THEREOF, AND NON-TRANSITORY COMPUTER READABLE MEDIUM

(71) Applicant: FUJIFILM Corporation, Tokyo (JP)

(72) Inventors: Saeko SASUGA, Tokyo (JP); Shuichi DOKKO, Chiba (JP)

(73) Assignee: FUJIFILM Corporation, Tokyo (JP)

(21) Appl. No.: 19/035,754

(22) Filed: Jan. 23, 2025

(30) Foreign Application Priority Data
Jan. 31, 2024 (JP) 2024-012552

(51) Int. Cl. A61B 8/00; A61B 8/08

(52) U.S. Cl. CPC A61B 8/445; A61B 8/0841

(57) ABSTRACT

Distal end coordinates of the puncture needle are acquired on the basis of a first image. Distal end coordinates of an ultrasound probe are acquired on the basis of the first image. Coordinates of a target region in an ultrasound image acquired by the ultrasound probe are acquired. Support information for supporting a puncture operation of the puncture needle is generated by using at least any one of the distal end coordinates of the puncture needle, the distal end coordinates of the ultrasound probe, or the coordinates of the target region. The support information is displayed on a display by being superimposed on the first image.

### CROSS-REFERENCE TO RELATED APPLICATIONS

This application claims priority under 35 U.S.C § 119 (a) to Japanese Patent Application No. 2024-012552 filed on 31 Jan. 2024. The above application is hereby expressly incorporated by reference, in its entirety, into the present application.

### BACKGROUND OF THE INVENTION

### 1. Field of the Invention

The present invention relates to a puncture support apparatus that supports an operation of a puncture needle used in laparoscopic surgery or the like, an operation method thereof, and a non-transitory computer readable medium.

### 2. Description of the Related Art

In the related art, although a laparotomy has been widely performed, laparoscopic surgery and robot surgery as less invasive surgery have been widely used. These surgical methods have an advantage that the amount of incision in a body of a patient is small because a small port hole is provided in the body and a surgical tool is inserted through the port hole.

In addition, as the surgical tool used in the laparoscopic surgery, there is an ultrasound probe for detecting a target region distributed in a depth direction in the body, such as a lesion portion. A distal end of the ultrasound probe used in the laparoscopic surgery is provided with, for example, a puncture hole or groove, and a puncture needle is passed through the puncture hole or groove of the ultrasound probe, so that it is possible to puncture an organ at an any angle (refer to, for example, JP2022-80023A (corresponding to US2022/0151707A1)). The puncture needle is used, for example, to inject indocyanine green (ICG) or indigo carmine into a portal vein blood vessel in order to determine a resection site of liver cancer surgery.

Note that, in U.S. Pat. No. 8,688,196B, a magnetic position sensor incorporated in a distal end portion of a puncture needle is detected using a magnetic field generation apparatus, and a relationship between the position of the puncture needle and the position of the ultrasound probe is detected, and the position of the puncture needle and the direction in which the puncture needle moves are graphically displayed on an ultrasound tomogram.

### SUMMARY OF THE INVENTION

In the laparoscopic surgery, as in the above-described advantage, the amount of incision can be reduced, but it is difficult to ascertain a positional relationship between a plurality of surgical tools from a camera image that observes the inside of the body, as compared with the laparotomy. In particular, in a case where a puncture needle is used to specify a surgical site of the liver as described above, it is difficult to appropriately insert the puncture needle only into the target blood vessel from among the blood vessels spread in the liver. Note that, in Masahiro Hasegawa, et al., “Accuracy of augmented reality with computed tomography-based navigation in total hip arthroplasty”, [online], [Nov. 6, 2023], Internet, <URL: https://www.ncbi.nlm.nih.gov/pmc/articles/PMC10481587/>, although there is a description regarding surgical navigation for identifying a relationship between the position of the pelvis and the position of the surgical tool with an AR marker, there is no description or suggestion regarding identification of the positional relationship between the puncture needle and the surgical tool such as another ultrasound probe.

An object of an exemplary embodiment of the invention is to provide a puncture support apparatus, an operation method thereof, and a non-transitory computer readable medium which can ascertain a positional relationship between a plurality of surgical tools in a body, such as an ultrasound probe and a puncture needle, and support an operation of a puncture needle.

A puncture support apparatus according to an aspect of the exemplary embodiment includes a processor, in which the processor acquires a first image acquired by an imaging apparatus that images a puncture needle or an ultrasound probe, acquires distal end coordinates of the puncture needle on the basis of the first image, acquires distal end coordinates of the ultrasound probe on the basis of the first image, acquires coordinates of a target region in an ultrasound image acquired by the ultrasound probe, generates support information for supporting a puncture operation of the puncture needle by using at least any one of the distal end coordinates of the puncture needle, the distal end coordinates of the ultrasound probe, or the coordinates of the target region, and displays the support information on a display by superimposing the support information on the first image.

It is preferable that the first image is a rigid endoscope image captured by a rigid endoscope that is the imaging apparatus, an intracorporeal puncture needle marker is attached to a portion of the puncture needle, which is inserted into a body, an intracorporeal probe marker is attached to a portion of the ultrasound probe, which is inserted into the body, the distal end coordinates of the puncture needle are calculated on the basis of the intracorporeal puncture needle marker included in the rigid endoscope image, and the distal end coordinates of the ultrasound probe are calculated on the basis of the intracorporeal probe marker included in the rigid endoscope image.

It is preferable that the first image is a camera image acquired by a camera that is installed outside a body and is the imaging apparatus, an extracorporeal puncture needle marker is attached to a portion of the puncture needle, which is positioned outside the body, an extracorporeal probe marker is attached to a portion of the ultrasound probe, which is positioned outside the body, the distal end coordinates of the puncture needle are calculated on the basis of the extracorporeal puncture needle marker included in the camera image, and the distal end coordinates of the ultrasound probe are calculated on the basis of the extracorporeal probe marker included in the camera image.

It is preferable that the processor acquires the first image and a second image that is different from the first image, from a plurality of imaging apparatuses, the first image is a rigid endoscope image captured by a rigid endoscope that is the imaging apparatus, the second image is a camera image acquired by a camera that is installed outside a body and is the imaging apparatus, an intracorporeal probe marker is attached to a portion of the ultrasound probe, which is inserted into the body, an extracorporeal puncture needle marker is attached to a portion of the puncture needle, which is positioned outside the body, the distal end coordinates of the ultrasound probe are calculated on the basis of the intracorporeal probe marker included in the rigid endoscope image, and the distal end coordinates of the puncture needle are calculated on the basis of the extracorporeal puncture needle marker included in the camera image.

It is preferable that the processor acquires the first image and a second image that is different from the first image, from a plurality of imaging apparatuses, the first image is a rigid endoscope image captured by a rigid endoscope as the imaging apparatus, the second image is a camera image acquired by a camera that is installed outside a body, as the imaging apparatus, an intracorporeal puncture needle marker is attached to a portion of the puncture needle, which is inserted into the body, an extracorporeal probe marker is attached to a portion of the ultrasound probe, which is positioned outside the body, the distal end coordinates of the ultrasound probe are calculated on the basis of the extracorporeal probe marker included in the camera image, and the distal end coordinates of the puncture needle are calculated on the basis of the intracorporeal puncture needle marker included in the rigid endoscope image.

It is preferable that an extracorporeal rigid endoscope marker is attached to a portion of the rigid endoscope, which is positioned outside the body, and distal end coordinates of the rigid endoscope are calculated on the basis of the extracorporeal rigid endoscope marker included in the camera image.

It is preferable that the processor generates a cross section guide that displays a position of an ultrasound cross section, as the support information, on the basis of the distal end coordinates of the ultrasound probe. It is preferable that the processor generates an extension line extending from a distal end of the puncture needle, as the support information, on the basis of the distal end coordinates of the puncture needle.

It is preferable that the processor generates a cross section guide that displays a position of an ultrasound cross section, as the support information, on the basis of the distal end coordinates of the ultrasound probe, and the processor makes a display aspect of the support information different between a case where the support information is on a front side of at least any one of the cross section guide, the ultrasound probe, or a cross section of the ultrasound image and a case where the support information overlaps or is on a back side of at least any one of the cross section guide, the ultrasound probe, or the cross section of the ultrasound image.

It is preferable that the processor generates a cross section guide that displays a position of an ultrasound cross section, as the support information, on the basis of the distal end coordinates of the ultrasound probe, and generates an extension line extending from a distal end of the puncture needle, as the support information, on the basis of the distal end coordinates of the puncture needle, and the processor makes a display aspect of the extension line different between a case where the extension line is on a front side of at least any one of the cross section guide, the ultrasound probe, or a cross section of the ultrasound image and a case where the extension line overlaps or is on a back side of at least any one of the cross section guide, the ultrasound probe, or the cross section of the ultrasound image.

It is preferable that the processor generates a puncture needle guide for guiding the puncture needle, in accordance with a position of a needle insertion hole of the ultrasound probe, as the support information, on the basis of the distal end coordinates of the ultrasound probe, and displays the puncture needle guide on the first image in a superimposed manner. It is preferable that the first image is a rigid endoscope image captured by a rigid endoscope that is the imaging apparatus. It is preferable that the first image is a camera image acquired by a camera that is installed outside a body and is the imaging apparatus.

It is preferable that the processor generates a puncture point that is a target insertion position of the puncture needle, as the support information, on the basis of the coordinates of the target region. It is preferable that the processor generates an extension line extending from a distal end of the puncture needle, as the support information, on the basis of the distal end coordinates of the puncture needle, and generates a puncture point that is a target insertion position of the puncture needle, as the support information, on the basis of the coordinates of the target region, and the processor generates a distance from the distal end of the puncture needle to the target region, as the support information in a case where the extension line overlaps the puncture point.

It is preferable that the processor generates a three-dimensional shape model of the target region, as the support information, on the basis of the ultrasound image and the coordinates of the target region, and generates a three-dimensional relationship from a distal end of the puncture needle to the target region, as the support information, on the basis of the distal end coordinates of the puncture needle and the coordinates of the target region.

An operation method of a puncture support apparatus including a processor according to another aspect of the exemplary embodiment includes, via the processor, a step of acquiring a first image acquired by an imaging apparatus that images a puncture needle or an ultrasound probe; a step of acquiring distal end coordinates of the puncture needle on the basis of the first image; a step of acquiring distal end coordinates of the ultrasound probe on the basis of the first image; a step of acquiring coordinates of a target region in an ultrasound image acquired by the ultrasound probe; a step of generating support information for supporting a puncture operation of the puncture needle by using at least any one of the distal end coordinates of the puncture needle, the distal end coordinates of the ultrasound probe, or the coordinates of the target region; and a step of displaying the support information on a display by superimposing the support information on the first image.

A non-transitory computer readable medium storing a computer-executable program according to another aspect of the exemplary embodiment causes a processor to execute a function of acquiring a first image acquired by an imaging apparatus that images a puncture needle or an ultrasound probe; a function of acquiring distal end coordinates of the puncture needle on the basis of the first image; a function of acquiring distal end coordinates of the ultrasound probe on the basis of the first image; a function of acquiring coordinates of a target region in an ultrasound image acquired by the ultrasound probe; a function of generating support information for supporting a puncture operation of the puncture needle by using at least any one of the distal end coordinates of the puncture needle, the distal end coordinates of the ultrasound probe, or the coordinates of the target region; and a function of displaying the support information on a display by superimposing the support information on the first image.

According to the exemplary embodiment of the invention, it is possible to ascertain the positional relationship between a plurality of surgical tools in the body, such as the ultrasound probe and the puncture needle, and to support the operation of the puncture needle.

### BRIEF DESCRIPTION OF THE DRAWINGS

FIG. 1 is a schematic diagram of a laparoscopic system.

FIG. 2 is an image diagram of an ultrasound image.

FIG. 3 is an explanatory diagram illustrating a first pattern of an acquisition pattern of distal end coordinates of an ultrasound probe and distal end coordinates of a puncture needle.

FIG. 4 is an explanatory diagram illustrating a second pattern of an acquisition pattern of distal end coordinates of an ultrasound probe and distal end coordinates of a puncture needle.

FIG. 5 is an explanatory diagram illustrating a third pattern of an acquisition pattern of distal end coordinates of an ultrasound probe and distal end coordinates of a puncture needle.

FIG. 6 is an explanatory diagram illustrating a fourth pattern of an acquisition pattern of distal end coordinates of an ultrasound probe and distal end coordinates of a puncture needle.

FIG. 7 is an explanatory diagram in a case where an extension line of a puncture needle overlaps a cross section guide in a first pattern of a generation pattern and a display pattern of support information.

FIG. 8 is an explanatory diagram in a case where an extension line of a puncture needle is on a front side of a cross section guide in a first pattern of a generation pattern and a display pattern of support information.

FIG. 9 is an explanatory diagram illustrating a second pattern of a generation pattern and a display pattern of support information.

FIG. 10 is an explanatory diagram illustrating a third pattern of a generation pattern and a display pattern of support information.

FIG. 11 is an explanatory diagram in a case where an extension line of a puncture needle does not overlap a puncture point in a fourth pattern of a generation pattern and a display pattern of support information.

FIG. 12 is an explanatory diagram in a case where an extension line of a puncture needle overlaps a puncture point in a fourth pattern of a generation pattern and a display pattern of support information.

FIG. 13 is an explanatory diagram illustrating a fifth pattern of a generation pattern and a display pattern of support information.

FIG. 14 is a flowchart illustrating the series of flows regarding support of an operation of a puncture needle.

FIG. 15 is an explanatory diagram in a case where an extension line of a puncture needle is on a front side of an ultrasound probe in a first pattern of a generation pattern and a display pattern of support information.

FIG. 16 is an explanatory diagram in a case where an extension line of a puncture needle overlaps an ultrasound probe in a first pattern of a generation pattern and a display pattern of support information.

FIG. 17 is an explanatory diagram in a case where an extension line of a puncture needle is on a front side of a cross section of an ultrasound image in a first pattern of a generation pattern and a display pattern of support information.

FIG. 18 is an explanatory diagram in a case where an extension line of a puncture needle overlaps a cross section of an ultrasound image in a first pattern of a generation pattern and a display pattern of support information.

### DESCRIPTION OF THE PREFERRED EMBODIMENTS

As illustrated in FIG. 1, a laparoscopic system 10 observes the inside of the body with a rigid endoscope 11, and detects a target region in a depth direction in the body, such as a lesion portion, using an ultrasound probe 12. In the laparoscopic system 10, a puncture needle 13 punctures a target region in the body at any angle. The puncture needle 13 is used, for example, to inject indocyanine green (ICG) or indigo carmine into a portal vein blood vessel in order to determine a resection site of liver cancer surgery. A camera 14 images the rigid endoscope 11, the ultrasound probe 12, or the puncture needle 13 used during laparoscopic surgery.

In the laparoscopic system 10, a puncture support apparatus 20 for supporting the insertion of the puncture needle 13 into the body is provided. A rigid endoscope image captured by the rigid endoscope 11 is transmitted to the puncture support apparatus 20 in a wired or wireless manner. An ultrasound image obtained by the ultrasound probe 12 is transmitted to the puncture support apparatus 20 in a wired or wireless manner. A camera image captured by the camera 14 is transmitted to the puncture support apparatus 20 in a wired or wireless manner. Note that the camera 14 may be a web camera or the like.

The puncture support apparatus 20 includes an image acquisition unit 21, a puncture needle coordinate acquisition unit 22, a probe coordinate acquisition unit 23, a target region coordinate acquisition unit 24, a support information generation unit 25, a display controller 26, and a display 27. The puncture support apparatus 20 is provided with a program memory (not illustrated) that stores a specific program. A controller (not illustrated) constituting a processor executes a specific program to realize functions of the image acquisition unit 21, the puncture needle coordinate acquisition unit 22, the probe coordinate acquisition unit 23, the target region coordinate acquisition unit 24, the support information generation unit 25, and the display controller 26.

The image acquisition unit 21 acquires a first image acquired by an imaging apparatus that images the puncture needle 13 or the ultrasound probe 12. The puncture needle coordinate acquisition unit 22 acquires the distal end coordinates of the puncture needle 13 on the basis of the first image. Since there are a plurality of acquisition patterns of the distal end coordinates of the puncture needle 13, the details will be described later. The probe coordinate acquisition unit 23 acquires the distal end coordinates of the ultrasound probe 12 on the basis of the first image. Since there are a plurality of acquisition patterns of the distal end coordinates of the ultrasound probe 12, the details will be described later.

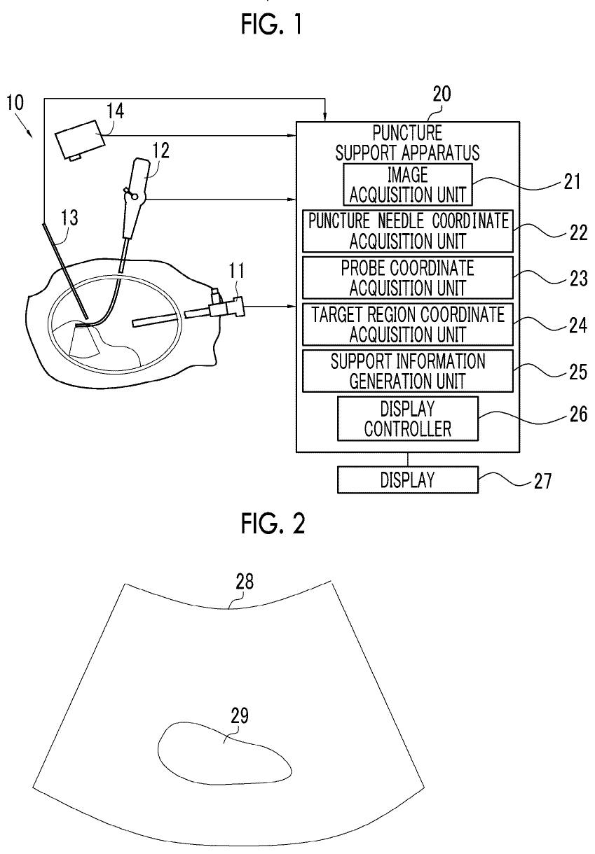

The target region coordinate acquisition unit 24 acquires coordinates of a target region in the ultrasound image acquired by the ultrasound probe 12. As illustrated in FIG. 2, the target region coordinate acquisition unit 24 acquires an image of a target region 29 such as a lesion portion, by performing target region detection processing on an ultrasound image 28. The support information generation unit 25 generates support information for supporting a puncture operation of the puncture needle 13, by using at least any one of the distal end coordinates of the puncture needle 13, the distal end coordinates of the ultrasound probe 12, or the coordinates of the target region. The display controller 26 displays the support information on the display by superimposing the support information on the first image. Since there are a plurality of generation patterns and display patterns of the support information, the details will be described later.

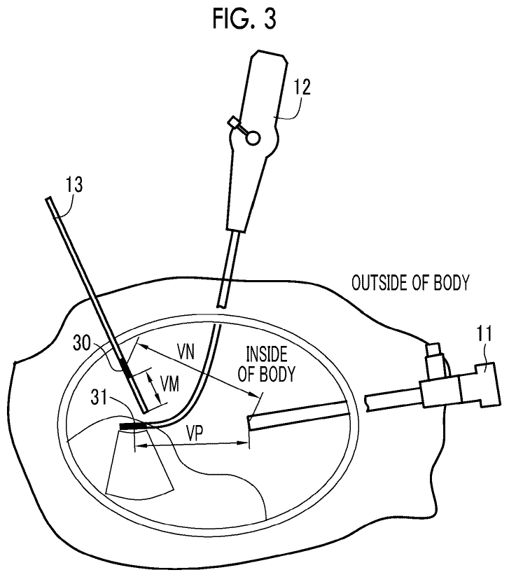

Regarding the acquisition pattern of the distal end coordinates of the ultrasound probe 12 and the distal end coordinates of the puncture needle 13, the following four patterns will be described. The first pattern is a pattern in which the distal end coordinates of the ultrasound probe 12 and the distal end coordinates of the puncture needle 13 are acquired from the image obtained in the body. As illustrated in FIG. 3, the puncture needle coordinate acquisition unit 22 acquires the distal end coordinates of the puncture needle 13 on the basis of the rigid endoscope image which is the first image. In order to acquire the distal end coordinates of the puncture needle 13, an intracorporeal puncture needle marker 30 is attached to a portion of the puncture needle 13, which is inserted into the body. The intracorporeal puncture needle marker 30 is provided at a portion separated from the distal end of the puncture needle 13 by a certain distance in the body. Therefore, the puncture needle coordinate acquisition unit 22 calculates the distal end coordinates of the puncture needle 13 from a vector VM between the intracorporeal puncture needle marker 30 and the distal end of the puncture needle 13, which is known, and a vector VN between the intracorporeal puncture needle marker 30 and the rigid endoscope 11, which is detected from the rigid endoscope image. Note that the intracorporeal puncture needle marker 30 is preferably an AR marker.

In addition, the probe coordinate acquisition unit 23 acquires the distal end coordinates of the ultrasound probe 12 on the basis of the rigid endoscope image which is the first image. In order to acquire the distal end coordinates of the ultrasound probe 12, an intracorporeal probe marker 31 is attached to a portion of the ultrasound probe 12 inserted into the body. The intracorporeal probe marker 31 is provided at the distal end of the ultrasound probe 12. Therefore, the distal end coordinates of the ultrasound probe 12 are calculated from a vector VP between the intracorporeal probe marker 31 and the rigid endoscope 11, which is detected from the rigid endoscope image. Note that the intracorporeal probe marker 31 is also preferably an AR marker.

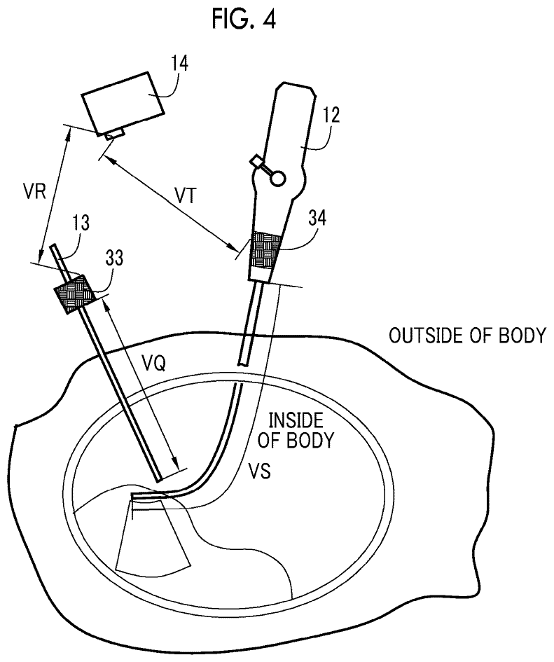

The second pattern is a pattern in which the distal end coordinates of the ultrasound probe 12 and the distal end coordinates of the puncture needle 13 are acquired from the image obtained outside the body. As illustrated in FIG. 4, the puncture needle coordinate acquisition unit 22 acquires the distal end coordinates of the puncture needle 13 on the basis of the camera image which is the first image. In order to acquire the distal end coordinates of the puncture needle 13, an extracorporeal puncture needle marker 33 is attached to a portion of the puncture needle 13 positioned outside the body. The extracorporeal puncture needle marker 33 is provided at a portion separated from the distal end of the puncture needle 13 by a certain distance outside the body. Therefore, the puncture needle coordinate acquisition unit 22 calculates the distal end coordinates of the puncture needle 13 from a vector VQ between the extracorporeal puncture needle marker 33 and the distal end of the puncture needle 13, which is known, and a vector VR between the extracorporeal puncture needle marker 33 and the camera 14, which is detected from the camera image. Note that the extracorporeal puncture needle marker 33 is preferably an AR marker.

In addition, the probe coordinate acquisition unit 23 acquires the distal end coordinates of the ultrasound probe 12 on the basis of the camera image which is the first image. In order to acquire the distal end coordinates of the ultrasound probe 12, an extracorporeal probe marker 34 is attached to a portion of the ultrasound probe 12 positioned outside the body. The extracorporeal probe marker 34 is provided at a specific portion of the ultrasound probe 12 outside the body. Therefore, the distal end coordinates of the ultrasound probe 12 are calculated from a vector VS between the extracorporeal probe marker 34 and the distal end of the ultrasound probe 12, which is known, and a vector VT between the extracorporeal probe marker 34 and the camera 14, which is detected from the camera image. Note that the extracorporeal probe marker 34 is also preferably an AR marker.

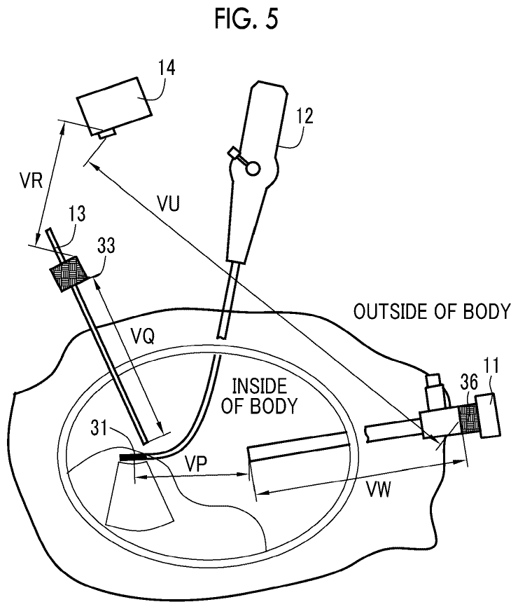

The third pattern is a pattern in which the distal end coordinates of the ultrasound probe 12 and the distal end coordinates of the puncture needle 13 are acquired from the images obtained inside and outside the body, and is different from the fourth pattern. As illustrated in FIG. 5, the puncture needle coordinate acquisition unit 22 calculates the distal end coordinates of the puncture needle 13 from the camera image which is a second image. A calculation method of the distal end coordinates of the puncture needle 13 is the same as that of the second pattern. In addition, it is preferable that the probe coordinate acquisition unit 23 calculates the distal end coordinates of the ultrasound probe 12 from the rigid endoscope image which is the first image. A calculation method of the distal end coordinates of the ultrasound probe 12 is the same as that of the first pattern. Note that, in a case where the distal end coordinates of the rigid endoscope 11 are acquired by the camera 14, it is preferable that an extracorporeal rigid endoscope marker 36 is attached to a portion of the rigid endoscope 11 positioned outside the body. In this case, the distal end coordinates of the rigid endoscope 11 are calculated on the basis of a vector VU between the camera 14 and the extracorporeal rigid endoscope marker 36, which is obtained from the camera image and a vector VW between the extracorporeal rigid endoscope marker 36 and the distal end of the rigid endoscope 11, which is known. The extracorporeal rigid endoscope marker 36 is also preferably an AR marker.

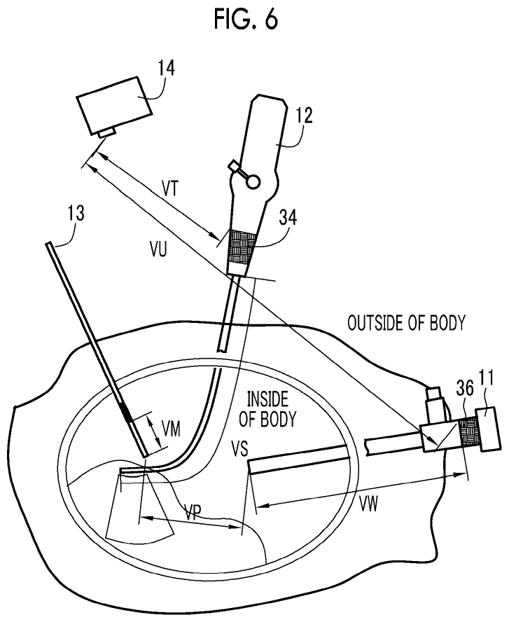

The fourth pattern is a pattern in which the distal end coordinates of the ultrasound probe 12 and the distal end coordinates of the puncture needle 13 are acquired from the images obtained inside and outside the body, and is different from the third pattern. As illustrated in FIG. 6, the puncture needle coordinate acquisition unit 22 calculates the distal end coordinates of the puncture needle 13 from the rigid endoscope image which is the first image. A calculation method of the distal end coordinates of the puncture needle 13 is the same as that of the first pattern. In addition, the probe coordinate acquisition unit 23 calculates the distal end coordinates of the ultrasound probe 12 from the camera image which is the second image. A calculation method of the distal end coordinates of the ultrasound probe 12 is the same as that of the second pattern. Note that, in a case where the distal end coordinates of the rigid endoscope 11 are acquired by the camera 14, as described above, it is preferable that the extracorporeal rigid endoscope marker 36 is attached to a portion of the rigid endoscope 11 positioned outside the body. In this case, the distal end coordinates of the rigid endoscope 11 are calculated on the basis of the vector VU between the camera 14 and the extracorporeal rigid endoscope marker 36, which is obtained from the camera image and the vector VW between the extracorporeal rigid endoscope marker 36 and the distal end of the rigid endoscope 11, which is known. The extracorporeal rigid endoscope marker 36 is also preferably an AR marker.

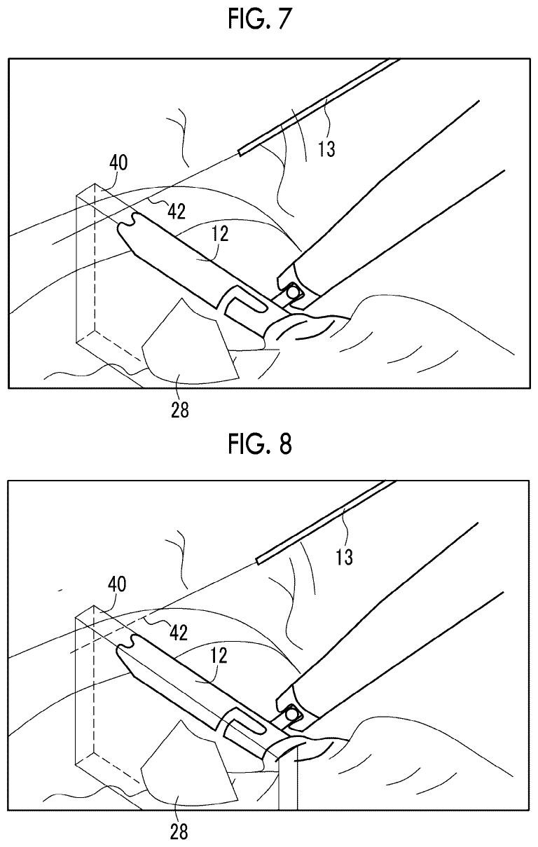

Regarding the generation pattern and the display pattern of the support information, the following five patterns will be described. In the first pattern, as illustrated in FIGS. 7 and 8, the support information generation unit 25 generates a cross section guide 40 that displays the position of an ultrasound cross section, as the support information, on the basis of the distal end coordinates of the ultrasound probe 12. The display controller 26 displays the ultrasound image 28 below the ultrasound probe 12 in a superimposed manner on the rigid endoscope image, and displays the cross section guide 40 in a superimposed manner on the rigid endoscope image. The cross section guide 40 is displayed in a superimposed manner in accordance with the shape of the distal end portion of the ultrasound probe 12, and is displayed extending slightly from the distal end portion of the ultrasound probe 12. In addition, the support information generation unit 25 generates an extension line extending from the distal end of the puncture needle, as the support information, on the basis of the distal end coordinates of the puncture needle. The display controller 26 displays an extension line 42 in a superimposed manner on the rigid endoscope image.

The display controller 26 makes the display aspect of the support information different between a case where the support information is on the front side of at least any one of the cross section guide 40, the ultrasound probe 12, or the cross section of the ultrasound image 28 and a case where the support information overlaps or is on the back side of at least any one of the cross section guide 40, the ultrasound probe 12, or the cross section of the ultrasound image 28. Specifically, the display controller 26 makes the display aspect of the extension line 42 different between a case where the extension line 42 is on the front side of at least any one of the cross section guide 40, the ultrasound probe 12, or the cross section of the ultrasound image 28 and a case where the extension line 42 overlaps at least any one of the cross section guide 40, the ultrasound probe 12, or the cross section of the ultrasound image 28 or is on the back side of at least any one of the cross section guide 40, the ultrasound probe 12, or the cross section of the ultrasound image 28. As a pattern for causing the display aspect of the extension line 42 to differ, for example, a part of the extension line 42 is made different by being displayed or not displayed. In this manner, the sense of depth can be realized by displaying or not displaying the support information such as the extension line 42.

In a case where the cross section guide 40 and the extension line 42 are generated, the display controller 26 makes the display aspect of the extension line 42 different between a case where the extension line 42 is on the front side of the cross section guide 40 and a case where the extension line 42 overlaps the cross section guide 40 or is on the back side of the cross section guide 40. Specifically, in a case where the extension line 42 overlaps the cross section guide 40 or is on the back side of the cross section guide 40, the display controller 26 does not display the portion of the extension line 42, which overlaps the cross section guide 40 or is on the back side of the cross section guide 40 (displays the portion by a dotted line in FIG. 8), and in a case where the extension line 42 is on the front side of the cross section guide 40, the display controller 26 displays the extension line (displays the extension line by a solid line in FIG. 7). As a result, the sense of depth can be given to the display aspect of the extension line. Since the entire extension line 42 is displayed in FIG. 7, it can be seen that the extension line 42 is on the front side of the cross section guide 40 without being superimposed on the cross section guide 40. Since a part of the extension line is not displayed in FIG. 8, it can be seen that the non-display portion overlaps the cross section guide 40. Note that the color of the extension line 42 may be made different between a case where the extension line 42 is on the front side of the cross section guide 40 and a case where the extension line 42 overlaps the cross section guide 40 or is on the back side of the cross section guide 40 (for example, the color of the extension line 42 is blue in a case where the extension line 42 overlaps the cross section guide 40 and the color of the extension line 42 is red in a case where the extension line 42 does not overlap the cross section guide 40).

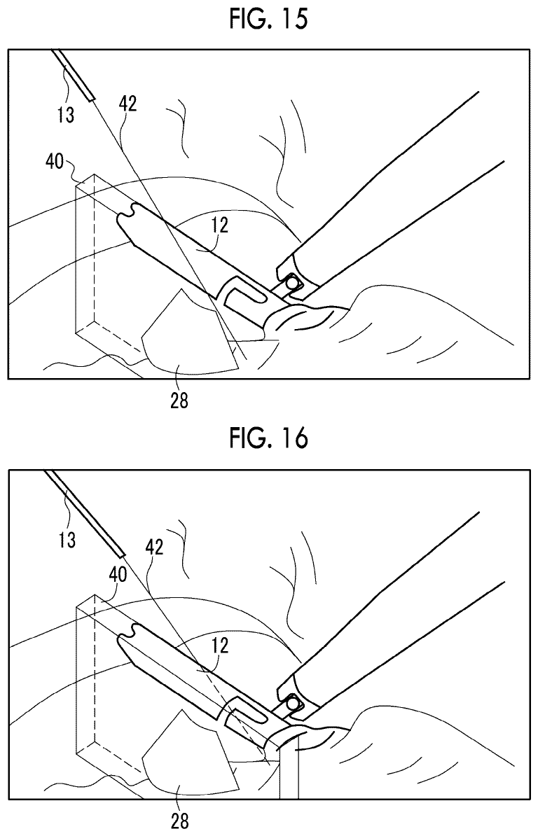

Note that the display controller 26 makes the display aspect of the extension line 42 different between a case where the extension line 42 is on the front side of the ultrasound probe 12 and a case where the extension line 42 overlaps the ultrasound probe 12 or is on the back side of the ultrasound probe 12. Specifically, as illustrated in FIG. 15, in a case where the extension line 42 is on the front side of the ultrasound probe 12, the display controller 26 displays the extension line (displays the extension line by a solid line in FIG. 15), and as illustrated in FIG. 16, in a case where the extension line 42 overlaps the ultrasound probe 12 or is on the back side of the ultrasound probe 12, the display controller 26 does not display the portion of the extension line 42, which overlaps the ultrasound probe 12 or is on the back side of the ultrasound probe 12 (displays the portion by a dotted line in FIG. 16).

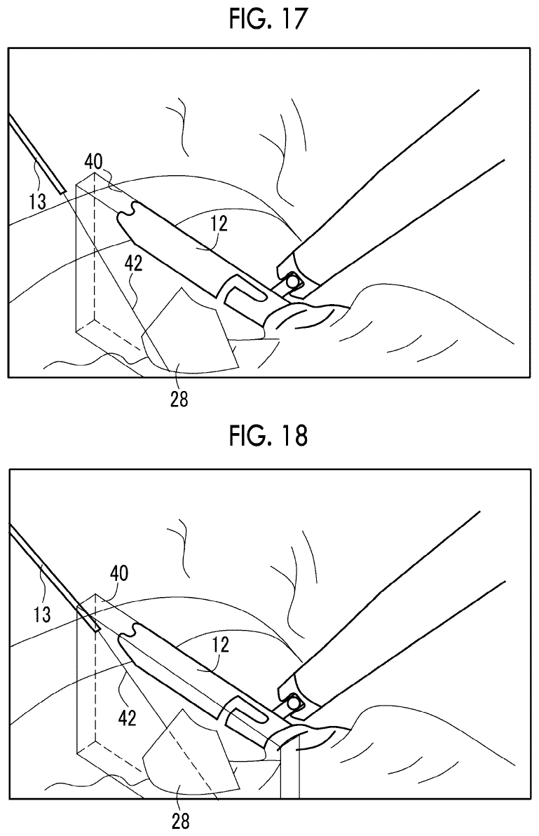

In addition, the display controller 26 makes the display aspect of the extension line 42 different between a case where the extension line 42 is on the front side of the cross section of the ultrasound image 28 and a case where the extension line 42 overlaps the cross section of the ultrasound image 28 or is on the back side of the cross section of the ultrasound image 28. Specifically, as illustrated in FIG. 17, in a case where the extension line 42 is on the front side of the cross section of the ultrasound image 28, the display controller 26 displays the extension line (displays the extension line by a solid line in FIG. 17), and as illustrated in FIG. 18, in a case where the extension line 42 overlaps the cross section of the ultrasound image 28 or is on the back side of the cross section of the ultrasound image 28, the display controller 26 does not display the portion of the extension line 42, which overlaps the cross section of the ultrasound image 28 or is on the back side of the cross section of the ultrasound image 28 (displays the portion by a dotted line in FIG. 18).

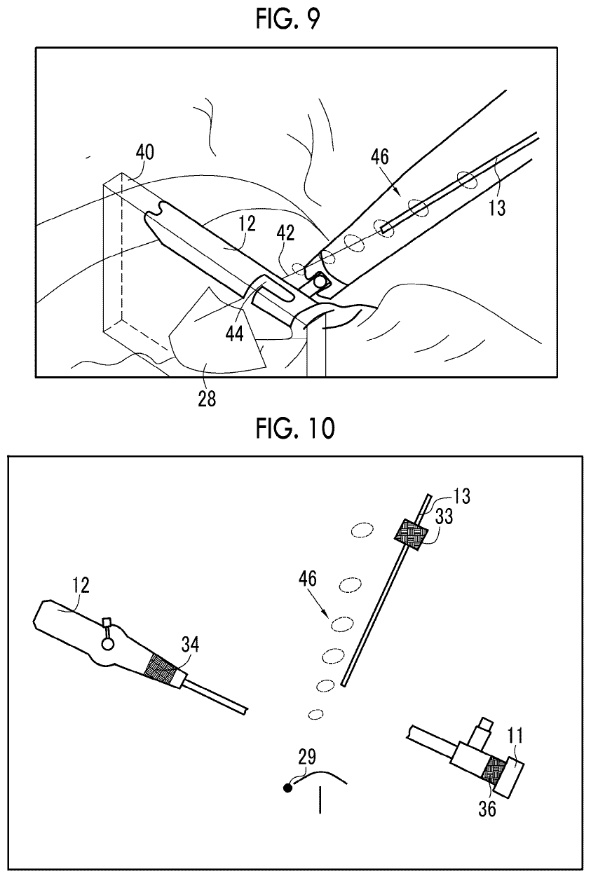

In the second pattern, as illustrated in FIG. 9, the support information generation unit 25 generates a puncture needle guide 46 for guiding the puncture needle 13 in accordance with the position of a needle insertion hole 44 of the ultrasound probe, as the support information, on the basis of the distal end coordinates of the ultrasound probe 12. The display controller 26 displays the ultrasound image below the ultrasound probe 12 in a superimposed manner on the rigid endoscope image (first image), and displays the puncture needle guide 46 in a superimposed manner on the rigid endoscope image (first image). The puncture needle guide 46 is formed of a plurality of ellipses, and the ellipses are provided at regular intervals toward the needle insertion hole 44. A user can reliably reach the needle insertion hole 44 by operating the puncture needle 13 to pass through the ellipses.

In the third pattern, as illustrated in FIG. 10, the image of the puncture needle guide 46 to be displayed in a superimposed manner in the second pattern is displayed in a superimposed manner on the camera image (first image) instead of the rigid endoscope image. As a result, it is possible to ascertain how much the current position of the puncture needle 13 is deviated from the target insertion position in the outside of the body. Note that, in the camera image, in order to display the coordinates of the target region 29, it is preferable to calculate the coordinates of the target region 29 using the ultrasound image even in the third pattern.

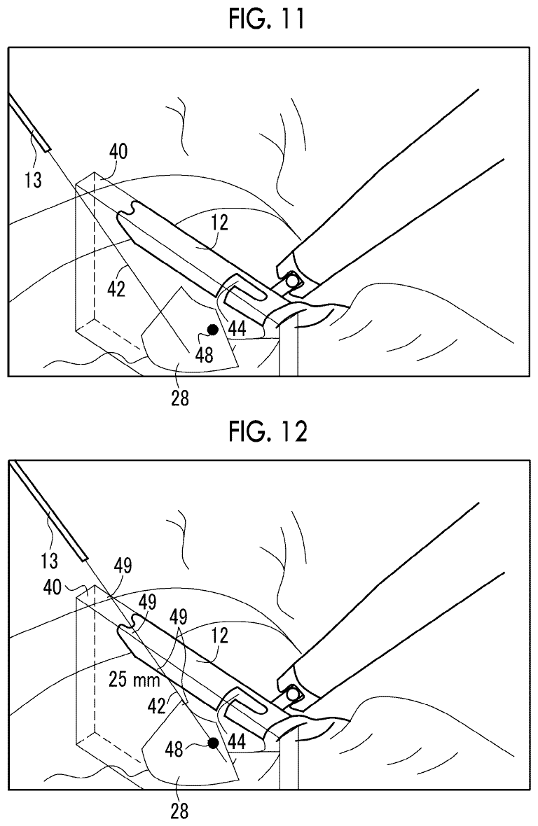

In the fourth pattern, as illustrated in FIG. 11, the support information generation unit 25 generates a puncture point 48, which is the target insertion position of the puncture needle 13, as the support information, on the basis of the coordinates of the target region. The display controller 26 displays the ultrasound image below the ultrasound probe 12 in a superimposed manner on the rigid endoscope image, and displays the puncture point 48 in the ultrasound image in a specific color and a specific shape (in FIG. 11, “O”). In addition, since the extension line 42 of the puncture needle 13 is also displayed in a superimposed manner, the positional relationship between the puncture needle 13 and the puncture point 48 can be ascertained.

In addition, in a case where the support information generation unit 25 generates the puncture point 48 and the extension line 42 of the puncture needle 13, as illustrated in FIG. 12, in a case where the extension line 42 overlaps the puncture point 48, the support information generation unit 25 calculates a distance from the distal end of the puncture needle 13 to the target region, as the support information, on the basis of the distal end coordinates of the puncture needle 13. The calculated distance is displayed on the display 27 by the display controller 26. In FIG. 12, “25 mm” is displayed as the distance, and a gradation 49 is provided at an interval of 5 mm.

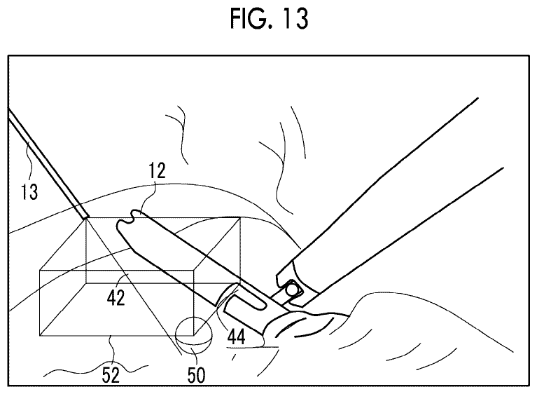

In the fifth pattern, the support information generation unit 25 generates a three-dimensional model of the target region, as the support information, on the basis of the ultrasound image 28 and the coordinates of the target region 29. As illustrated in FIG. 13, the display controller 26 displays, as the three-dimensional model of the target region 29, a shape model 50 representing the target region 29 on the display 27. In addition, the support information generation unit 25 generates a three-dimensional relationship from the puncture needle 13 to the target region 29, as the support information, on the basis of the distal end coordinates of the puncture needle 13 and the coordinates of the target region 29. As illustrated in FIG. 13, the display controller 26 displays a lattice-shaped rectangular parallelepiped 52 as the three-dimensional relationship. Since the three-dimensional relationship such as the distance and the direction between the puncture needle 13 and the target region 29 can be ascertained by the lattice-shaped rectangular parallelepiped 52, the user can easily operate the puncture needle 13.

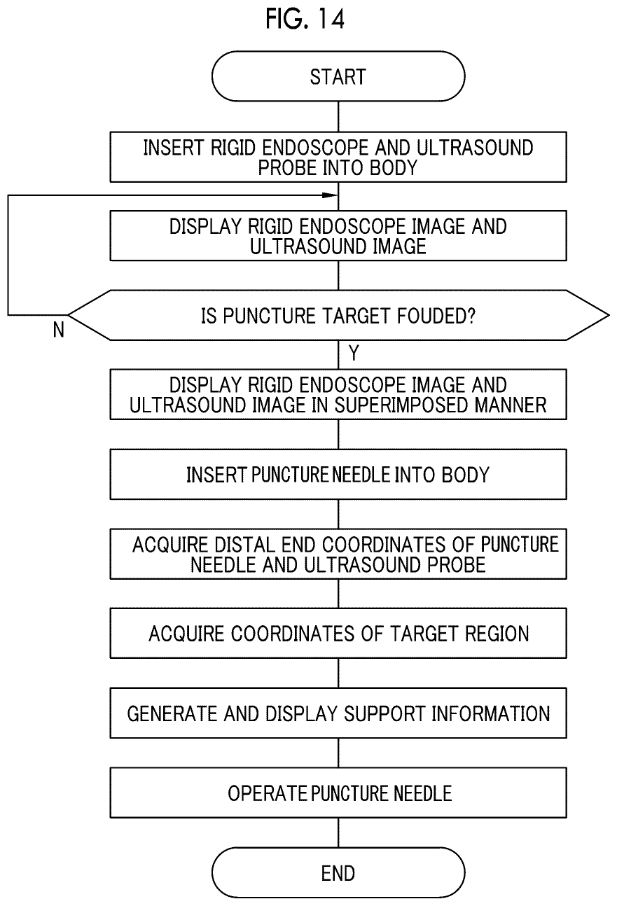

Next, the series of flows for supporting the operation of the puncture needle 13 in a case of performing surgery on the liver will be described with reference to the flowchart of FIG. 14. First, by using a trocar or the like, the rigid endoscope 11 and the ultrasound probe 12 are inserted into the body from the abdominal cavity. The ultrasound probe 12 is provided with any one of the intracorporeal probe marker 31 or the extracorporeal probe marker 34. The ultrasound image obtained by the ultrasound probe 12 and the rigid endoscope image obtained by the rigid endoscope 11 are displayed separately on the display 27. The user searches for a puncture target by placing the ultrasound probe 12 against the liver while observing the display 27. In a case where the puncture target is found by the search, a user interface (not illustrated) connected to the puncture support apparatus 20 is operated to switch to a superimposed display mode in which an ultrasound image is displayed on a rigid endoscope image in a superimposed manner. As a result, the image in which the ultrasound image is displayed in a superimposed manner on a puncture target portion in the rigid endoscope image is displayed on the display 27.

Next, the user inserts the puncture needle 13 into the body while observing the display 27. The puncture needle 13 is provided with any one of the intracorporeal puncture needle marker 30 or the extracorporeal puncture needle marker 33. The puncture needle coordinate acquisition unit 22 acquires the distal end coordinates of the puncture needle 13 on the basis of the first image acquired by the imaging apparatus that images the puncture needle 13 or the ultrasound probe 12. The probe coordinate acquisition unit 23 acquires the distal end coordinates of the ultrasound probe 12 on the basis of the first image. The target region coordinate acquisition unit 24 acquires the coordinates of the target region in the ultrasound image.

The support information generation unit 25 generates the support information by using at least any one of the distal end coordinates of the puncture needle 13, the distal end coordinates of the ultrasound probe 12, or the coordinates of the target region. The support information is displayed on the display 27 by being displayed on the first image in a superimposed manner. The user performs the operation of the puncture needle 13 while observing the support information displayed on the display 27.

Specifically, in a case where the first image is the rigid endoscope image, the puncture needle 13 is provided with the intracorporeal puncture needle marker, and the ultrasound probe 12 is provided with the intracorporeal puncture needle marker 30, the distal end coordinates of the puncture needle 13 and the distal end coordinates of the ultrasound probe 12 can be obtained from the rigid endoscope image. Then, as the support information, the extension line 42 extending from the distal end of the puncture needle 13 is generated on the basis of the distal end coordinates of the puncture needle 13, and is displayed on the display 27. In addition, as the support information, the cross section guide 40 that displays the position of the ultrasound cross section is generated on the basis of the distal end coordinates of the ultrasound probe 12, and is displayed on the display 27.

The user operates the puncture needle 13 in the body while observing the extension line 42 and the cross section guide 40 displayed on the display. The extension line 42 is also moved following the operation of the puncture needle 13. Then, in a case where the puncture needle 13 is moved to an appropriate position, such as a case where the extension line 42 overlaps the target region, the puncture needle 13 punctures the target site.

In the embodiment described above, the hardware structures of processing units that execute various kinds of processing, such as the image acquisition unit 21, the puncture needle coordinate acquisition unit 22, the probe coordinate acquisition unit 23, the target region coordinate acquisition unit 24, the support information generation unit 25, and the display controller 26 are various processors as described below. The various processors include a central processing unit (CPU) that is a general-purpose processor which functions as various processing units by executing software (program), a graphical processing unit (GPU), a programmable logic device (PLD) that is a processor capable of changing a circuit configuration after manufacture, such as a field programmable gate array (FPGA), and a dedicated electric circuit that is a processor having a circuit configuration specifically designed to execute various kinds of processing.

One processing unit may be composed of one of the various processors or may be composed of a combination of two or more processors of the same type or different types (for example, a combination of a plurality of FPGAs, a combination of a CPU and an FPGA, or a combination of a CPU and a GPU). In addition, one processor may constitute a plurality of processing units. As an example where a plurality of processing units are configured by one processor, first, there is an aspect where one processor is configured by a combination of one or more CPUs and software as typified by a computer, such as a client or a server, and this processor functions as a plurality of processing units. Second, there is an aspect where a processor fulfilling the functions of the entire system including a plurality of processing units by one integrated circuit (IC) chip as typified by a system on chip (SoC) or the like is used. As described above, the various processing units are configured using one or more of the various processors as a hardware structure.

Furthermore, the hardware structures of the various processors are more specifically electrical circuitry in a form in which circuit elements, such as semiconductor elements, are combined. In addition, a hardware structure of the storage unit is a storage device such as a hard disc drive (HDD) and a solid state drive (SSD).

### EXPLANATION OF REFERENCES

10: laparoscopic system
11: rigid endoscope
12: ultrasound probe
13: puncture needle
14: camera
20: puncture support apparatus
21: image acquisition unit
22: puncture needle coordinate acquisition unit
23: probe coordinate acquisition unit
24: target region coordinate acquisition unit
25: support information generation unit
26: display controller
27: display
28: ultrasound image
29: target region
30: intracorporeal puncture needle marker
31: intracorporeal probe marker
33: extracorporeal puncture needle marker
34: extracorporeal probe marker
36: extracorporeal rigid endoscope marker
40: cross section guide
42: extension line
44: needle insertion hole
46: puncture needle guide
48: puncture point
49: gradation
50: shape model
52: lattice-shaped rectangular parallelepiped

What is claimed is:

1. A puncture support apparatus comprising:
a processor,
wherein the processor is configured to:
acquire a first image acquired by an imaging apparatus that images a puncture needle or an ultrasound probe;
acquire distal end coordinates of the puncture needle on the basis of the first image;
acquire distal end coordinates of the ultrasound probe on the basis of the first image;
acquire coordinates of a target region in an ultrasound image acquired by the ultrasound probe;
generate support information for supporting a puncture operation of the puncture needle, by using at least any one of the distal end coordinates of the puncture needle, the distal end coordinates of the ultrasound probe, or the coordinates of the target region; and
display the support information on a display by superimposing the support information on the first image.

2. The puncture support apparatus according to claim 1,
wherein the first image is a rigid endoscope image captured by a rigid endoscope that is the imaging apparatus,
an intracorporeal puncture needle marker is attached to a portion of the puncture needle, which is inserted into a body,
an intracorporeal probe marker is attached to a portion of the ultrasound probe, which is inserted into the body,
the distal end coordinates of the puncture needle are calculated on the basis of the intracorporeal puncture needle marker included in the rigid endoscope image, and
the distal end coordinates of the ultrasound probe are calculated on the basis of the intracorporeal probe marker included in the rigid endoscope image.

3. The puncture support apparatus according to claim 1,
wherein the first image is a camera image acquired by a camera that is installed outside a body and is the imaging apparatus,
an extracorporeal puncture needle marker is attached to a portion of the puncture needle, which is positioned outside the body,
an extracorporeal probe marker is attached to a portion of the ultrasound probe, which is positioned outside the body,
the distal end coordinates of the puncture needle are calculated on the basis of the extracorporeal puncture needle marker included in the camera image, and
the distal end coordinates of the ultrasound probe are calculated on the basis of the extracorporeal probe marker included in the camera image.

4. The puncture support apparatus according to claim 1,
wherein the processor is configured to acquire the first image and a second image that is different from the first image, from a plurality of imaging apparatuses,
the first image is a rigid endoscope image captured by a rigid endoscope that is the imaging apparatus,
the second image is a camera image acquired by a camera that is installed outside a body and is the imaging apparatus,
an intracorporeal probe marker is attached to a portion of the ultrasound probe, which is inserted into the body,
an extracorporeal puncture needle marker is attached to a portion of the puncture needle, which is positioned outside the body,
the distal end coordinates of the ultrasound probe are calculated on the basis of the intracorporeal probe marker included in the rigid endoscope image, and
the distal end coordinates of the puncture needle are calculated on the basis of the extracorporeal puncture needle marker included in the camera image.

5. The puncture support apparatus according to claim 1,
wherein the processor is configured to acquire the first image and a second image that is different from the first image, from a plurality of imaging apparatuses,
the first image is a rigid endoscope image captured by a rigid endoscope as the imaging apparatus,
the second image is a camera image acquired by a camera that is installed outside a body, as the imaging apparatus,
an intracorporeal puncture needle marker is attached to a portion of the puncture needle, which is inserted into the body,
an extracorporeal probe marker is attached to a portion of the ultrasound probe, which is positioned outside the body,
the distal end coordinates of the ultrasound probe are calculated on the basis of the extracorporeal probe marker included in the camera image, and
the distal end coordinates of the puncture needle are calculated on the basis of the intracorporeal puncture needle marker included in the rigid endoscope image.

6. The puncture support apparatus according to claim 4,
wherein an extracorporeal rigid endoscope marker is attached to a portion of the rigid endoscope, which is positioned outside the body, and
distal end coordinates of the rigid endoscope are calculated on the basis of the extracorporeal rigid endoscope marker included in the camera image.

7. The puncture support apparatus according to claim 1,
wherein the processor is configured to generate a cross section guide that displays a position of an ultrasound cross section, as the support information, on the basis of the distal end coordinates of the ultrasound probe.

8. The puncture support apparatus according to claim 1,
wherein the processor is configured to generate an extension line extending from a distal end of the puncture needle, as the support information, on the basis of the distal end coordinates of the puncture needle.

9. The puncture support apparatus according to claim 1,
wherein the processor is configured to:
generate a cross section guide that displays a position of an ultrasound cross section, as the support information, on the basis of the distal end coordinates of the ultrasound probe; and
make a display aspect of the support information different, between a case where the support information is on a front side of at least any one of the cross section guide, the ultrasound probe, or a cross section of the ultrasound image, and a case where the support information overlaps or is on a back side of at least any one of the cross section guide, the ultrasound probe, or the cross section of the ultrasound image.

10. The puncture support apparatus according to claim 1,
wherein the processor is configured to:
generate a cross section guide that displays a position of an ultrasound cross section, as the support information, on the basis of the distal end coordinates of the ultrasound probe;
generate an extension line extending from a distal end of the puncture needle, as the support information, on the basis of the distal end coordinates of the puncture needle; and
make a display aspect of the extension line different, between a case where the extension line is on a front side of at least any one of the cross section guide, the ultrasound probe, or a cross section of the ultrasound image, and a case where the extension line overlaps or is on a back side of at least any one of the cross section guide, the ultrasound probe, or the cross section of the ultrasound image.

11. The puncture support apparatus according to claim 1,
wherein the processor is configured to generate a puncture needle guide for guiding the puncture needle, in accordance with a position of a needle insertion hole of the ultrasound probe, as the support information, on the basis of the distal end coordinates of the ultrasound probe, and display the puncture needle guide on the first image in a superimposed manner.

12. The puncture support apparatus according to claim 10,
wherein the first image is a rigid endoscope image captured by a rigid endoscope that is the imaging apparatus.

13. The puncture support apparatus according to claim 10,
wherein the first image is a camera image acquired by a camera that is installed outside a body and is the imaging apparatus.

14. The puncture support apparatus according to claim 1,
wherein the processor is configured to generate a puncture point that is a target insertion position of the puncture needle, as the support information, on the basis of the coordinates of the target region.

15. The puncture support apparatus according to claim 1,
wherein the processor is configured to:
generate an extension line extending from a distal end of the puncture needle, as the support information, on the basis of the distal end coordinates of the puncture needle;
generate a puncture point that is a target insertion position of the puncture needle, as the support information, on the basis of the coordinates of the target region; and
generate a distance from the distal end of the puncture needle to the target region, as the support information in a case where the extension line overlaps the puncture point.

16. The puncture support apparatus according to claim 1,
wherein the processor is configured to:
generate a three-dimensional shape model of the target region, as the support information, on the basis of the ultrasound image and the coordinates of the target region; and
generate a three-dimensional relationship from a distal end of the puncture needle to the target region, as the support information, on the basis of the distal end coordinates of the puncture needle and the coordinates of the target region.

17. An operation method of a puncture support apparatus including a processor, the operation method comprising:
via the processor,
acquiring a first image acquired by an imaging apparatus that images a puncture needle or an ultrasound probe;
acquiring distal end coordinates of the puncture needle on the basis of the first image;
acquiring distal end coordinates of the ultrasound probe on the basis of the first image;
acquiring coordinates of a target region in an ultrasound image acquired by the ultrasound probe;
generating support information for supporting a puncture operation of the puncture needle by using at least any one of the distal end coordinates of the puncture needle, the distal end coordinates of the ultrasound probe, or the coordinates of the target region; and
displaying the support information on a display by superimposing the support information on the first image.

18. A non-transitory computer readable medium for storing a computer-executable program, the computer-executable program causing a processor to execute:
a function of acquiring a first image acquired by an imaging apparatus that images a puncture needle or an ultrasound probe;
a function of acquiring distal end coordinates of the puncture needle on the basis of the first image;
a function of acquiring distal end coordinates of the ultrasound probe on the basis of the first image;
a function of acquiring coordinates of a target region in an ultrasound image acquired by the ultrasound probe;
a function of generating support information for supporting a puncture operation of the puncture needle by using at least any one of the distal end coordinates of the puncture needle, the distal end coordinates of the ultrasound probe, or the coordinates of the target region; and
a function of displaying the support information on a display by superimposing the support information on the first image.
